(19) United States
(12) Patent Application Publication
(10) Pub. No.: US 2007/0009458 A1
(43) Pub. Date: Jan. 11, 2007

(54) Lipophilic personal care composition

(75) Inventors: Hisatoshi Masuda, Moriyama (JP); Mayu Ishigami, Ashiya (JP)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(73) Assignee: The Procter & Gamble Company

(21) Appl. No.: 11/482,022

(22) Filed: Jul. 6, 2006

Related U.S. Application Data
(60) Provisional application No. 60/697,851, filed on Jul. 8, 2005.

(51) Int. Cl. A61K 8/89

(52) U.S. Cl. 424/64; 424/70.12

(57) ABSTRACT

Disclosed is a lipophilic personal care composition comprising by weight: (a) from about 5% to about 50% of an N-long chain acyl acidic amino acid ester of sterol which is liquid at 25° C. having formula (I):
XOC(O)—(CH₂)ₙ—CH(NHCOR)—C(O)OY (I) wherein one of X and Y is an ester residue of sterol, and the other is selected from H, a liquid alkyl or alkenyl of 8-30 carbons, and ester residue of monohydric solid alcohol of 12-38 carbons; COR is a long chain acyl group of 8-22 carbons; and n is an integer of 1 or 2, and (b) from about 0.5% to about 15% of an acryl alkyl silicone copolymer having a melting point of no more than about 40° C.

### CROSS REFERENCE TO RELATED APPLICATION

This application claims the benefit of U.S. Provisional Application No. 60/697,851, filed on Jul. 8, 2005.

### FIELD OF THE INVENTION

The present invention relates to lipophilic personal care compositions comprising a N-long chain acyl acidic amino acid ester or diester of sterol in liquid form, and an acryl alkyl silicone copolymer having a melting point of no more than about 40° C. The composition is particularly useful for making cosmetic compositions such as lipstick, lipgloss, foundations, and creams.

### BACKGROUND

Recently, the lipstick market has been segmented into those consumers who seek wear benefit, moisturizing benefit, and balance of wear and moisturizing benefits. Lipsticks are primarily made of lipophilic or hydrophobic materials. Lipsticks designed for providing a moisturizing benefit to the lips have a tendency of further containing polar solvents, or other moisturizing components which are more or less hydrophilic. The use of association structures and gel structures has been suggested to bind such polar solvents in the lipophilic matrix of the lipstick, such as in PCT publication WO 02/26198. While such lipstick compositions provide a favorable moisturizing benefit to the lips, some compositions were unsatisfactory in physical stability, and some others required uncommon equipment or extra processing steps.

Meanwhile, consumers who seek moisturizing benefit also have a tendency of desiring ideal texture of the lips, namely soft, plumpy look texture. Such texture improvement may or may not be achieved only by use of polar solvents.

Cosmetic compositions utilizing a N-long chain acyl acidic amino acid ester or diester of sterol, and an acryl alkyl silicone copolymer is disclosed, for example, in Japanese publication A-2003-113021. While it is disclosed that such composition provides good feel at use, shine, wear, and moisturization, further improvement in feel at use is desired.

Based on the foregoing, there is a need for a lipophilic personal care composition which provides improved moisturization and improved texture of the skin, while also having good physical stability. There is also a need for a personal care composition which provides a smooth feel upon use. There is also a need for a personal care composition which can be manufactured by utilizing commonly used equipment and processes.

### SUMMARY

The present invention is directed to a lipophilic personal care composition comprising by weight:
(a) from about 5% to about 50% of an N-long chain acyl acidic amino acid ester of sterol which is liquid at 25° C. having formula (I):
   XOC(O)—(CH₂)ₙ—CH(NHCOR)—C(O)OY (I)
wherein one of X and Y is an ester residue of sterol, and the other is selected from H, a liquid alkyl or alkenyl of 8-30 carbons, and ester residue of monohydric solid alcohol of 12-38 carbons; COR is a long chain acyl group of 8-22 carbons; and n is an integer of 1 or 2; and
(b) from about 0.5% to about 15% of an acryl alkyl silicone copolymer having a melting point of no more than about 40° C.

The present invention is further directed to a lipophilic personal care composition which satisfies the need for improved texture of the skin, while also being moisturizing on the skin, provides a smooth feel upon use, and having good physical stability.

These and other features, aspects, and advantages of the present invention will become evident to those skilled in the art from a reading of the present disclosure with the appended claims.

### DETAILED DESCRIPTION

The following is a list of definitions for terms used herein.

“Comprising” means that other steps and other ingredients which do not affect the end result can be added. This term encompasses the terms “consisting of” and “consisting essentially of”.

All percentages are by weight of total composition unless specifically stated otherwise.

All cited references are incorporated herein by reference in their entireties. Citation of any reference is not an admission regarding any determination as to its availability as prior art to the claimed invention.

All ratios are weight ratios unless specifically stated otherwise.

The present invention, in its product and process aspects, is described in detail as follows.

N-Long Chain Acyl Acidic Amino Acid Ester or Diester of Sterol

The composition of the present invention comprises a N-long chain acyl acidic amino acid ester of sterol which is liquid at 25° C. having formula (I):
XOC(O)—(CH₂)ₙ—CH(NHCOR)—C(O)OY (I)
wherein one of X and Y is an ester residue of sterol, and the other is selected from H, a liquid alkyl or alkenyl of 8-30 carbons, and ester residue of monohydric solid alcohol of 12-38 carbons; COR is a long chain acyl group of 8-22 carbons; and n is an integer of 1 or 2. The ester may be a monoester or diester. Preferably, one of X and Y is an ester residue of phytosterol, cholesterol, lanosterol, stigmasterol, its hydrogenates, and its derivatives, more preferably phytosterol, and the other is selected from 2-octyldodecyl alcohol, isostearyl alcohol, oleyl alcohol, cetyl alcohol, benenyl alcohol, and mixtures thereof. The N-long chain acyl acidic amino acid ester of sterol useful herein is liquid at 25° C., is effective in alleviating damaged skin, has high water holding capability, provide a favorable feel to the skin, and is compatible with many oily materials useful for making lipophilic personal care compositions. It has been surprisingly found that, when N-long chain acyl acidic amino acid ester of sterol is combined with an acryl alkyl silicone copolymer, a lipophilic composition having a smooth feel upon use is obtained.

The N-long chain acyl acidic amino acid ester of sterol is comprised by weight of the entire composition at from about 5% to about 50%. In the particularly preferred lipstick embodiment, the N-long chain acyl acidic amino acid ester of sterol is comprised by weight of the entire composition at from about 5% to about 25%, preferably from about 10% to about 20%.

Commercially available N-long chain acyl acidic amino acid ester of sterol include: phytosteryl/octyldodecyl lauroyl glutamate with tradename ELDEW PS-203 and cholesteryl/octyldodecyl lauroyl glutamate with tradename ELDEW CL-202, both available from Ajinomoto.

Acryl Alkyl Silicone Copolymer

The composition of the present invention comprises an acryl alkyl silicone copolymer having a melting point of no more than about 40° C. The acryl alkyl silicone copolymers useful herein include those that are graft copolymers of an (meth)acrylic main chain and siloxane side chains, wherein the (meth)acrylic main chain has attached to it an alkyl chain of 16-22 carbons. The acryl alkyl silicone copolymer is believed to provide smoothness to the composition.

The acryl alkyl silicone copolymer is comprised by weight of the entire composition at from about 0.5% to about 15%, preferably from about 1% to about 10%.

Commercially available acryl alkyl silicone copolymer include: acrylates/stearyl acrylate/dimethicone methacrylate copolymer with tradename KP-561P available from ShinEtsu Silicone.

Lipophilic Composition

The composition of the present invention is made of single continuous lipophilic phase. The present composition may be anhydrous. While there could be a small amount of water included in the composition, such water is within the amount that could be completely solubilized in the lipophilic components of the composition. For example, N-long chain acyl acidic amino acid ester of sterol has a certain water holding capability. The present invention excludes compositions that are emulsions.

The lipophilic personal care composition of the present invention may take various consumer product forms, such as lipstick, lipgloss, foundation, skin cream, antiperspirant, hair treatment cream, ointment, and others. The present composition is particularly useful for making cosmetic compositions such as lipstick, lipgloss, foundations, and creams.

The composition of the present invention may contain further lipophilic components for making the remainder of the composition. Such components can be any that are useful for personal care products, and which characterize the product. Nonlimiting examples of the other lipophilic components include polyglycerin fatty acid ester, dimer acid ester, low viscosity vegetable oil, and other nonvolatile oils. For providing the preferred cosmetic compositions of the present invention, components such as solid wax, color powders, skin feel improving powders, skin benefit agents, UV absorbers, and other components may be included. One highly preferred embodiment of the present invention is lipstick. The present lipstick composition comprises solid wax for solidifying the composition, and color powders for providing color to the lips.

In one aspect of the present invention, the lipophilic composition is substantially free of polar solvents. What is meant by polar solvents, are hydrophilic liquid solvents such as water, and monohydric and polyhydric alcohols. By providing the composition substantially free of polar solvents, the composition is also free from the need of providing special structures in the lipophilic continuous phase for carrying the polar solvent. Thus, the present composition substantially free of polar solvents has good physical stability. In the highly preferred lipstick embodiment, good physical stability means prevention of sweating and color stability. Moreover, the present composition substantially free of polar solvents has good moisturizing despite lack of polar solvents.

In another aspect of the present invention, the lipophilic composition comprises polar solvents. The polar solvents may be used as solvent for dissolving water-soluble skin benefit agents to be incorporated in the lipophilic composition.

In yet another aspect of the present invention, the lipophilic composition is substantially free of surfactants. Surfactants herein include emulsifiers. By providing the composition substantially free of surfactants, the composition has reduced risk of causing irritation to the skin or scalp.

By “substantially free” what is meant is that none of such component is actively included in the composition. However, the present invention does not exclude the use of components which may carry an insignificant amount of such component as an impurity or byproduct of another component.

The lipophilic composition of the present invention may be suitably manufactured by utilizing commonly used equipment and processes known in the art. The present composition may be made by conveniently mixing the components in a tank with heat added as necessary for fluidizing solid components. Heat sensitive components may be added after the mixture is cooled.

Polyglycerin Fatty Acid Ester

The composition of the present invention may further comprise a polyglycerin fatty acid ester having a polyglycerin of 2-15 units and at least 5 fatty acid residues attached thereto, wherein the fatty acid residue has 18 to 24 carbons. The polyglycerin fatty acid ester useful herein is liquid at room temperature, has high water holding capability, and is compatible with many oily materials useful for making lipophilic personal care compositions.

In the particularly preferred lipstick embodiment, the polyglycerin fatty acid ester may be comprised by weight of the entire composition at from about 5% to about 30%, preferably from about 10% to about 25%.

Polyglycerin fatty acid esters useful herein include esters made of polyglyceryl-2, polyglyceryl-4, polyglyceryl-6, or polyglyceryl-10; with at least 5 isostearate residues.

Commercially available polyglycerin fatty acid esters useful herein include polyglyceryl-10 nonaisostearate with tradename S-FACE IS-1009P available from Sakamoto Pharmaceuticals.

Dimer Acid Ester

The composition of the present invention may further comprise a dimer acid ester. The dimer acid ester may be a monoester or diester of dimer acid, wherein at least one group is esterified with a sterol. The dimer acid ester may also be a diester of dimer acid esterified with fatty alcohol. The sterol may be phytosterol, cholesterol, lanosterol, stigmasterol, or its hydrogenates. The remaining group or both groups may be esterified with a straight chain alcohol having 1-34 carbons, a branched chain alcohol having 3-34 carbons, or an unsaturated alcohol having 6-34 carbons. Preferably, the dimer acid ester is a sterol ester which is liquid in room temperature.

In the particularly preferred lipstick embodiment, the dimer acid ester may be comprised by weight of the entire composition at from about 5% to about 25%, preferably from about 10% to about 20%.

Commercially available dimer acid esters include: phytosteryl isostearyl dimer dilinoleate with tradename LUSPLAN PI-DA, dimer dilinoleyl hydrogenated rosinate with tradename LUSPLAN DD-DHR, and dimer dilinoleyl dimer dilinoleate: with tradenames LUSPLAN DD-DA5 and LUSPLAN DD-DA7, all available from Nippon Fine Chemical Co., Ltd.

Low Viscosity Vegetable Oil

The composition of the present invention may further comprise a low viscosity vegetable oil having a viscosity of no more than about 300 mPas, preferably no more than about 200 mPas. Low viscosity vegetable oils useful herein are any that are extracted from vegetable origin mainly comprising glyceride esters, and its hydrogenated products. Those having a relatively low viscosity are particularly useful herein for providing the desired fresh feel for the entire lipophilic composition. Non-limited examples of low viscosity vegetable oils useful herein are: sweet almond oil, rapeseed oil, fennel oil, olive oil, orange oil, canola oil, matricaria flower oil, cucumber oil, aleurites moluccana seed oil, cinnamon oil, corn oil, arctium lappa seed oil, sesame seed oil, rice bran oil, rice germ oil, camellia kissi seed oil, safflower seed oil, shea butter oil, job's tears seed oil, peppermint oil, soybean oil, tea seed oil, camellia japonica seed oil, rosa canina fruit oil, apricot kernel oil, palm kernel oil, palm oil, hybrid safflower seed oil, hybrid sunflower oil, peanut oil, sunflower seed oil, grape seed oil, jojoba oil, macadamia integrifolia nut oil, meadowfoam seed oil, coconut oil, eucalyptus oil, eucalyptus clobulus leaf oil, rosemary leaf oil, evening primrose oil, its hydrogenated products, and mixtures thereof. Preferred for use herein are macadamia integrifolia nut oil, meadowfoam seed oil, olive oil, rosa canina fruit oil, sunflower seed oil, coconut oil, sweet almond oil, shea butter oil, and mixtures thereof.

In the particularly preferred lipstick embodiment, the low viscosity vegetable oil may be comprised by weight of the entire composition at from about 5% to about 20%, preferably from about 10% to about 15%.

Commercially available low viscosity vegetable oil include macadamia integrifolia nut oil with tradename FLORAMAC HAWAIIAN MACADAMIA available from Floratech, meadowfoam seed oil with tradename CROPURE MDF available from Croda, olive oil with tradename CROPURE OL available from Croda, jojoba oil with tradename Jojoba Oil available from Mitsuba Trading, and safflower seed oil with tradename Safflower Seed Oil available from NOF Corporation.

Other Non-Volatile Oil

The composition of the present invention may further comprise other non-volatile oils suitable for personal use. The non-volatile oil may be comprised by weight of the entire composition at from about 1% to about 50%, preferably from about 5% to about 30%.

Non-volatile oils useful herein are, for example, tridecyl isononanoate, isostearyl isostearate, isocetyl isosteatrate, isopropyl isostearate, isodecyl isonoanoate, cetyl octanoate, isononyl isononanoate, diisopropyl myristate, isocetyl myristate, isotridecyl myristate, isopropyl myristate, isostearyl palmitate, isocetyl palmitate, isodecyl palmitate, isopropyl palmitate, octyl palmitate, caprylic/capric acid triglyceride, glyceryl tri-2-ethylhexanoate, diglyceryl sebacate, neopentyl glycol di(2-ethyl hexanoate), diisopropyl dimerate, tocopherol, tocopherol acetate, castor oil, turtle oil, mink oil, eggyolk oil, castor oil, glycerol trioctanate, glycerol triisopalmitate, polyglyceryl-2 triisostearate, trimethylolpropane triisostearate, isopropyl myristate, glycerol tri-2-ethylhexanoate, pentaerythritol tetra-2-ethylhexanoate, lanolin, liquid lanolin, liquid paraffin, squalane, vaseline, shea butter, and mixtures thereof. Commercially available oils include, for example, tridecyl isononanoate with tradename Crodamol TN available from Croda, Hexalan available from Nisshin Oil Mills, Ltd., tocopherol acetates available from Eisai, polyglyceryl-2 triisostearate with tradename COSMOL 43V available from Nisshin Oil Mills, Ltd., and shea butter with tradename CROPURE SB available from Croda.

Non-volatile oils useful herein also include polyalkyl or polyaryl siloxanes with the following structure (II) 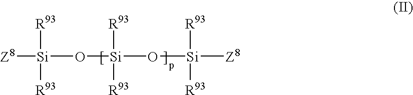
wherein R⁹³ is alkyl or aryl, and p is an integer from about 7 to about 8,000. Z⁸ represents groups which block the ends of the silicone chains. The alkyl or aryl groups substituted on the siloxane chain (R⁹³) or at the ends of the siloxane chains Z⁸ can have any structure as long as the resulting silicone remains fluid at room temperature, is dispersible, is neither irritating, toxic nor otherwise harmful when applied to the skin, is compatible with the other components of the composition, and is chemically stable under normal use and storage conditions. Suitable Z⁸ groups include hydroxy, methyl, methoxy, ethoxy, propoxy, and aryloxy. The two R⁹³ groups on the silicon atom may represent the same group or different groups. Preferably, the two R⁹³ groups represent the same group. Suitable R⁹³ groups include methyl, ethyl, propyl, phenyl, methylphenyl and phenylmethyl. The preferred silicone compounds are polydimethylsiloxane, polydiethylsiloxane, and polymethylphenylsiloxane. Polydimethylsiloxane, which is also known by CTFA name dimethicone, is especially preferred. These silicone compounds are available, for example, from the General Electric Company in their Viscasil® and SF 96 series, and from Dow Corning in their Dow Corning 200 series.

Polyalkylaryl siloxane fluids can also be used and include, for example, polymethylphenylsiloxanes. These siloxanes also known by CTFA name phenyl trimethicone are available, for example, from the General Electric Company as SF 1075 methyl phenyl fluid, from Dow Corning as 556 Cosmetic Grade Fluid, or from ShinEtsu Silicone as Silicone Oil KF-56.

Non-volatile oils also useful herein are the various grades of mineral oils. Mineral oils are liquid mixtures of hydrocarbons that are obtained from petroleum. Specific examples of suitable hydrocarbons include paraffin oil, mineral oil, dodecane, isododecane, hexadecane, isohexadecane, eicosene, isoeicosene, tridecane, tetradecane, polybutene, polyisobutene, and mixtures thereof.

Solid Wax

The composition of the present invention may further comprise a solid wax. For providing solid personal care compositions such as lipstick, solid foundations and antiperspirant sticks, solid wax is typically included. The amount of the solid wax is controlled to provide the desired hardness and strength to the product. In the particularly preferred lipstick embodiment, the solid wax is comprised by weight of the entire composition at from about 5% to about 20%, preferably from about 8% to about 15%.

The solid waxes useful herein are paraffin wax, microcrystalline wax, ozokerite wax, ceresin wax, carnauba wax, candelilla wax, eicosanyl behenate, polyethylene wax, and mixtures thereof. A mixture of waxes is preferably used.

Commercially available solid waxes useful herein include: Candelilla wax NC-1630 available from Cerarica Noda Co., Ltd., Ozokerite Wax SP-1021 available from Strahl & Pitsh, eicosanyl behenate available from Cas Chemical, microcrystalline wax with tradename MULTIWAX 180-M Yellow available from Witco Chemical, and polyethylene wax with tradename PERFORMALENE PL available from New Phase Technology.

Color Powders

The composition of the present invention may further comprise color powders. Herein, color powders include those which are white or nearly transparent. For providing the preferred cosmetic compositions, the color powder component is comprised by weight of the entire composition at from about 1% to about 50%, preferably from about 2% to about 45%. The amount and type of powders are selected depending on the desired characteristic of the product, for example, shade, coverage, UV protection benefit, and various skin feel.

The materials useful herein are clay mineral powders such as talc, mica, sericite, silica, magnesium silicate, synthetic fluorphlogopite, calcium silicate, aluminum silicate, bentonite and montomorillonite; pearl pigments such as alumina, barium sulfate, calcium secondary phosphate, calcium carbonate, titanium oxide, finely divided titanium oxide, zirconium oxide, zinc oxide, hydroxy apatite, iron oxide, iron titate, ultramarine blue, Prussian blue, chromium oxide, chromium hydroxide, cobalt oxide, cobalt titanate, titanium oxide coated mica; organic powders such as polyester, polyethylene, polystyrene, methyl metharylate resin, cellulose, 12-nylon, 6-nylon, styrene-acrylic acid copolymers, poly proprylene, vinyl chloride polymer, tetrafluoroethylene polymer, boron nitride, fish scale guanine, laked tar color dyes, and laked natural color dyes.

A certain percentage of spherical powders can be used. In the preferred foundation embodiments, the materials may be selected depending on the oil absorbing capability of the powders.

Hydrophobically treated powders can also be used. Such hydrophobically treated powders are made by treating the base material, as above, with a hydrophobical treatment agent, including: silicone such as Methicone, Dimethicone and perfluoroalkylsilane; fatty material such as stearic acid; metal soap such as aluminium dimyristate; aluminium hydrogenated tallow glutamate, hydrogenated lecithin, lauroyl lysine, aluminium salt of perfluoroalkyl phosphate, and mixtures thereof.

Polar Solvent

In certain embodiments, the composition of the present invention may further comprise a polar solvent. The polar solvent provides moisturizing benefit to the skin. The polar solvent also serves as solvent for incorporating water-soluble skin benefit agents which otherwise would not be compatible in the lipophilic carrier base.

For providing cosmetic compositions, when included, the polar solvent is comprised by weight of the entire composition at from about 0.01% to about 10%, preferably from about 1% to about 8%.

Polar solvents useful herein include water, polyhydric alcohols such as glycerin, 1,3-butylene glycol, propylene glycol, hexylene glycol, propane diol, ethylene glycol, diethylene glycol, dipropylene glycol, diglycerin, sorbitol, and other sugars which are in liquid form at ambient temperature. Also useful herein are water soluble alkoxylated nonionic polymers such as polyethylene glycol.

Commercially available polar solvents herein include: glycerin available from Asahi Denka; propylene glycol with tradename LEXOL PG-865/855 available from Inolex, 1,2-PROPYLENE GLYCOL USP available from BASF; 1,3-butylene glycol available from Daisel Kagaku Kogyo; dipropylene glycol with the same tradename available from BASF; diglycerin with tradename DIGLYCEROL available from Solvay GmbH.

Water-Soluble Skin Benefit Agent

The composition of the present invention may further comprise a water-soluble skin benefit agent in an amount soluble in the polar solvent above, preferably from about 0.1% to about 10%, more preferably from about 1% to about 5%.

Water-soluble skin benefit agents useful herein include niacinamide, panthenol, bacterial cultured mediums, allantoin, sodium lactate, PCA soda, amino acids, urea, sodium hyaluronate, chondroitin sulfate, collagen, elastin, pectin, carageenan, sodium alginate, trehalose, tuberose saccharide, chitin derivatives, chitosan derivatives, and mixtures thereof.

Commercially available water soluble skin benefit agents include niacinamide and panthenol by Roche, sodium hyaluronate with tradenames ACTIMOIST available from Active Organics, AVIAN SODIUM HYALURONATE series available from Intergen, and HYALURONIC ACID Na available from Ichimaru Pharcos.

Additional Components

The composition of the present invention may include other additional components, which may be selected by the artisan according to the desired characteristics of the final product and which are suitable for rendering the composition more cosmetically or aesthetically acceptable or to provide them with additional usage benefits. Such additional components generally are used individually at levels of no more than about 5% by weight of the composition.

The composition of the present invention may further contain a nonvolatile dispersed silicone usually referred to as silicone gum. The term “silicone gum”, as used herein, means a polyorganosiloxane material having a viscosity at 25° C. of greater than or equal to 1,000,000 mPa·s. Silicone gums are believed to provide wearability improvement such as long-lasting effect. The “silicone gums” will typically have a mass molecular weight in excess of about 200,000, generally between about 200,000 and about 1,000,000. Specific examples include polydimethylsiloxane, poly(dimethylsiloxane methylvinylsiloxane) copolymer, poly(dimethylsiloxane diphenylsiloxane methylvinylsiloxane) copolymer and mixtures thereof. Commercially available silicone gums are described in General Electric Silicone Rubber Product Data Sheets as SE 30, SE 33, SE 54 and SE 76.

The composition of the present invention may further contain a silicone resin, which are highly crosslinked polymeric siloxane systems. Silicone resins are believed to enhance spreadability and improve the feel to the skin. The crosslinking is introduced through the incorporation of tri-functional and tetra-functional silanes with mono-functional or di-functional, or both, silanes during manufacture of the silicone resin. As is well understood in the art, the degree of crosslinking that is required in order to result in a silicone resin will vary according to the specific silane units incorporated into the silicone resin. In general, silicone materials which have a sufficient level of trifunctional and tetrafunctional siloxane monomer units, and hence, a sufficient level of crosslinking, such that they dry down to a rigid, or hard, film are considered to be silicone resins. The ratio of oxygen atoms to silicon atoms is indicative of the level of crosslinking in a particular silicone material. Silicone materials which have at least about 1.1 oxygen atoms per silicon atom will generally be silicone resins herein. Preferably, the ratio of oxygen:silicon atoms is at least about 1.2:1.0. Silanes used in the manufacture of silicone resins include monomethyl-, dimethyl-, trimethyl-, monophenyl-, diphenyl-, methylphenyl-, monovinyl-, and methylvinylchlorosilanes, and tetrachlorosilane, with the methyl substituted silanes being most commonly utilized. Preferred are crosslinked silicone powders with tradenames Trefil E-505C, Trefil E-506C, and 9506 Powder; suspensions of silicone elastomer powders with tradenames BY29-119 and BY29-122; and silicone compound emulsions with tradenames SH5500, SC5570, and SM 5571; all available from Dow Corning.

Other useful silicone resins are silicone resin powders such as the material given the CTFA designation polymethylsilsequioxane, which is commercially available as Tospearl™ from Toshiba Silicones.

Silicone materials and silicone resins in particular, can conveniently be identified according to a shorthand nomenclature system well known to those skilled in the art as the “MDTQ” nomenclature. Under this system, the silicone is described according to the presence of various siloxane monomer units which make up the silicone. Briefly, the symbol M denotes the mono-functional unit (CH₃)₃SiO_{0.5}; D denotes the difunctional unit (CH₃)₂SiO; T denotes the trifunctional unit (CH₃)SiO_{1.5}; and Q denotes the quadri- or tetra-functional unit SiO2. Primes of the unit symbols, e.g., M′, D′, T′, and Q′ denote substituents other than methyl, and must be specifically defined for each occurrence. Typical alternate substituents include groups such as vinyl, phenyl, amino, hydroxyl, etc. The molar ratios of the various units, either in terms of subscripts to the symbols indicating the total number of each type of unit in the silicone, or an average thereof, or as specifically indicated ratios in combination with molecular weight, complete the description of the silicone material under the MDTQ system. Higher relative molar amounts of T, Q, T′ and/or Q′ to D, D′, M and/or or M′ in a silicone resin is indicative of higher levels of crosslinking. As discussed before, however, the overall level of crosslinking can also be indicated by the oxygen to silicon ratio.

The silicone resins for use herein which are preferred are MQ, MT, MTQ, MQ and MDTQ resins. Thus, the preferred silicone substituent is methyl. Especially preferred are MQ resins wherein the M:Q ratio is from about 0.5:1.0 to about 1.5:1.0 and the average molecular weight of the resin is from about 1000 to about 10,000. Commercially available MQ resins are, for example, trimethyl siloxy silicate with tradename BY11-018 available from Dow Corning.

The composition of the present invention may further contain a water-soluble polymer. It is believed that water-soluble polymers provide long-lasting effect. Useful water-soluble polymers include sodium carboxymethyl cellulose, polyvinyl pyrrolidone, polyvinyl alcohol, xanthan gum, agar, pulleran, bentonite, and mixtures thereof. Commercially available water-soluble polymers include the Carbopol series available from B. F. Goodrich Company, and PVP K-30 available from G.A.F. Chemicals.

The composition of the present invention may further contain an oil swelling clay material which functions as a thickener for the composition. Oil swelling clay materials useful herein include hectorite, bentonite, montmorillonite, and bentone clays which have been modified to be compatible with oil. Preferably, the modification is quaternization with an ammonium compound. Preferable oil swelling clay materials include quaternary ammonium modified hectorite. Commercially available oil swelling clay materials include benzyldimethyl stearyl ammonium hectorite with tradename Bentone 38 CG OR available from Rheox. Inc.

Other components which can be formulated into the compositions of the present invention are; emollient mixtures by the tradename MAXI-LIP containing octyl palmitate, tribehenin, sorbitan isostearate and palmitoyl oligopeptide available from Sederma, preservatives such as benzyl alcohol, methyl paraben, propyl paraben, imidazolidinyl area, and EDTA and its salts, perfumes, ultraviolet and infrared screening and absorbing agents such as ethylhexyl methoxycinnamate, and others.

### EXAMPLES

The following examples further describe and demonstrate the preferred embodiments within the scope of the present invention. The examples are given solely for the purpose of illustration, and are not to be construed as limitations of the present invention since many variations thereof are possible without departing from its spirit and scope.

### Examples 1-5

The following make-up compositions are formed by the following components using the method of preparation described herein:

| No | Component | Ex. 1 | Ex. 2 | Ex. 3 | Ex. 4 | Ex. 5 |
|---|---|---|---|---|---|---|
| 1 | Shea Butter *1 | 5 | | | 5 | 2 |
| 2 | Acrylates/Stearyl Acrylate/ | 2 | 2 | 2 | 5 | 1 |
| | Dimethicone Methacrylate | | | | | |
| | Copolymer *2 | | | | | |
| 3 | Emollient mixture of Octyl | 0.5 | 0.5 | 0.5 | 1.0 | |
| | Palmitate, Tribehenin, Sorbitan | | | | | |
| | Isostearate and Palmitoyl | | | | | |
| | Oligopeptide *3 | | | | | |
| 4 | Phenyl Trimethicone *4 | 10 | 10 | 10 | 20 | 10 |
| 5 | Phytosteryl/Octyldodecyl | 12.5 | 12.5 | 17.5 | | 10.0 |
| | Lauroyl Glutamate *5 | | | | | |
| 6 | Cholesteryl/Octyldodecyl | | 5 | | 12.5 | 3 |
| | Lauroyl Glutamate *6 | | | | | |
| 7 | Phytosteryl Isostearyl Dimer | 15 | 15 | 15 | 15 | |
| | Dilinoleate *7 | | | | | |
| 8 | Macadamia Integrifolia Nut | 5 | 5 | 5 | 5 | 5 |
| | Oil *8 | | | | | |
| 9 | Limnathes Alba (Meadowfoam) | 5 | 5 | 5 | 5 | 5 |
| | Seed Oil *9 | | | | | |
| 10 | Ozokerite Wax *10 | 2 | 2 | 2 | 2 | 1 |
| 11 | Microcrystalline Wax *11 | 2 | 2 | 2 | 2 | 1 |
| 12 | Candelilla Wax *12 | 0.5 | 0.5 | 0.5 | 0.5 | |
| 13 | Polyethylene Wax *13 | 5.5 | 5.5 | 5.5 | 5.5 | 5.0 |
| 14 | Polyglyceryl-2 | 7.5 | 7.5 | 7.5 | 7.5 | 5 |
| | Triisostearate *14 | | | | | |
| 15 | Polyglyceryl-10 | 17 | 17 | 17 | 13.5 | 11.5 |
| | Nonaisostearate *15 | | | | | |
| 16 | Preservatives | 0.5 | 0.5 | 0.5 | 0.5 | 0.5 |
| 17 | Powder Pigments | 10 | 10 | 10 | | 40 |

Definitions of Components
*1 Shea Butter: CROPURE SB available from Croda
*2 Acrylates/Stearyl Acrylate/Dimethicone Methacrylate Copolymer: KP-561P available from ShinEtsu Silicone
*3 Emollient mixture of Octyl Palmitate, Tribehenin, Sorbitan Isostearate and Palmitoyl Oligopeptide: MAXI-LIP available from Sederma
*4 Phenyl Trimethicone: Silicone Oil KF-56 available from ShinEtsu Silicone
*5 Phytosteryl/Octyldodecyl Lauroyl Glutamate: ELDEW PS-203 available from Ajinomoto Ltd.
*6 Cholesteryl/Octyldodecyl Lauroyl Glutamate: ELDEW CL-202 available from Ajinomoto Ltd.
*7 Phytosteryl Isostearyl Dimer Dilinoleate: LUSPLAN PI-DA available from Nippon Fine Chemical Co., Ltd.
*8 Macadamia Integrifolia Nut Oil: FLORAMAC HAWAIIAN MACADAMIA available from FLORATECH
*9 Limnathes Alba (Meadowfoam) Seed Oil: CROPURE MDF available from Croda
*10 Ozokerite Wax: Ozokerite Wax SP-1021 available from Strahl & Pitsh
*11 Microcrystalline Wax: MULTIWAX 180-M Yellow available from Witco Chemical
*12 Candelilla Wax: Candelilla Wax NC-1630 available from Cerarica Noda Co., Ltd.
*13 Polyethylene Wax: PERFORMALENE PL available from New Phase Technology
*14 Polyglyceryl-2 Triisostearate: COSMOL 43V available from Nisshin Oil Mills, Ltd.
*15 Polyglyceryl-10 Nonaisostearate: S-FACE IS-1009P available from Sakamoto Pharmaceuticals.

Method of Preparation

The cosmetic compositions of Examples 1-5 are suitably prepared as follows: First, a mixture of component numbers 1 through 16 are heated to disperse using a Propeller Mixer at 95° C. in a sealed tank. After all components are melted, cool the mixture to 85° C. After that, component number 17 is added, and the mixture is dispersed at 85° C. Finally, the dispersion is filled in an air-tight container and allowed to cool to room temperature.

Examples 1 through 3 provide lipsticks, Example 4 provides lipgloss, and Example 5 provides foundation. These embodiments represented by the previous examples have many advantages. For example, the cosmetic compositions provide improved texture of the lips or skin, while also being moisturizing on the lips or skin, provides smooth feel upon use, and have good physical stability.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A lipophilic personal care composition comprising by weight:
(a) from about 5% to about 50% of an N-long chain acyl acidic amino acid ester of sterol which is liquid at 25° C. having formula (I):
XOC(O)—(CH₂)ₙ—CH(NHCOR)—C(O)OY (I)
wherein one of X and Y is an ester residue of sterol, and the other is selected from H, a liquid alkyl or alkenyl of 8-30 carbons, and ester residue of monohydric solid alcohol of 12-38 carbons; COR is a long chain acyl group of 8-22 carbons; and n is an integer of 1 or 2; and
(b) from about 0.5% to about 15% of an acryl alkyl silicone copolymer having a melting point of no more than about 40° C.

2. The lipophilic personal care composition of claim 1 wherein the composition is substantially free of polar solvents.

3. The lipophilic personal care composition of claim 1 wherein the composition is substantially free of surfactants.

4. The lipophilic personal care composition of claim 1 for use as a lipstick, the lipstick comprising by weight:
(a) from about 5% to about 25% of the N-long chain acyl acidic amino acid ester of sterol; and
(b) from about 0.5% to about 15% of the acryl alkyl silicone copolymer.

5. The lipstick composition of claim 4 further comprising a solid wax.

6. The lipstick composition of claim 4 further comprising a coloring powder.
